(19) United States
(12) Patent Application Publication
(10) Pub. No.: US 2019/0177478 A1
(43) Pub. Date: Jun. 13, 2019

(54) Process for preparing polymers from monomers comprising laurolactam

(71) Applicant: Evonik Degussa GmbH, Essen (DE)

(72) Inventors: Kévin MICOINE, Herten (DE); Kristina ZUMBANSEN, Essen (DE); Franz-Erich BAUMANN, Dülmen (DE); Marcus Matthias STERGAR, Herten (DE); Andreas PAWLIK, Recklinghausen (DE); Christoph HILLER, Dülmen (DE); Mirko MICHEL, Dortmund (DE); Christoph KOOPMANN, Essen (DE)

(73) Assignee: Evonik Degussa GmbH, Essen (DE)

(21) Appl. No.: 16/216,143

(22) Filed: Dec. 11, 2018

(30) Foreign Application Priority Data
Dec. 13, 2017 (EP) 17206928.8

(51) Int. Cl. C08G 69/16; C07D 255/02

(52) U.S. Cl. CPC C08G 69/16; C07D 255/02

(57) ABSTRACT

Polymers can be prepared from monomers comprising laurolactam, by a process including a. Beckmann rearrangement of cyclododecanone oxime to give laurolactam in the presence of a Beckmann rearrangement catalyst, b. removal of impurities from the laurolactam to obtain purified laurolactam, and c. polymerization of monomers comprising purified laurolactam. For avoidance of discolouration or yellowing under ageing conditions, prior to the polymerization, polycyclic substances containing 24 carbon atoms and at least one heteroatom selected from oxygen and nitrogen and having a molar mass between 300 and 380 g/mol are limited to 500 ppm, based on laurolactam.

### CROSS-REFERENCE TO RELATED APPLICATIONS

The present application claims priority to EP17206928.8, filed 13 Dec. 2017, which is incorporated herein by reference in its entirety.

### BACKGROUND OF THE INVENTION

### Field of the Invention

The present invention relates to a process for preparing polymers from monomers comprising laurolactam and optionally further comonomers, and to polymers which are obtained from the process.

### Description of Related Art

Known polymers composed of monomers comprising laurolactam are, for example, nylon-12, copolyamides containing laurolactam such as caprolactam-laurolactam copolyamide or block copolymers such as polyether-block-amides (PEBA). Laurolactam is obtained, for example, from cyclododecanone oxime. Cyclododecanone oxime can be prepared by the reaction of cyclododecanone and hydroxylamine in an organic solvent (EP 2551261). In addition, cyclododecanone oxime can be obtained by the reaction of cyclododecanone with hydrogen peroxide and ammonia in the presence of a titanium silicalite catalyst (EP 1316545=US 2003/100795, EP 1663955=US 2008/249300), Cyclododecanone oxime can alternatively be obtained by photooximation of cyclododecane (EP 0993438=U.S. Pat. No. 6,197,999).

Laurolactam can be prepared by means of Beckmann rearrangement of cyclododecanone oxime to give laurolactam in the presence of a strong acid, cyanuric chloride, phosphorus trichloride or thionyl chloride (Ullmann's encyclopedia 2009, 11, 33-36; EP 2013162=US 2009/306367; EP2123635). The strong acid used may, for example, be sulfuric acid.

The colour of the polymer is very important for many applications. The prior art discloses that impurities in laurolactam promote the yellowing of corresponding polyamides. In EP 1773765 (US 2008/071081), lactams are prepared by means of photooximation, giving chlorinated impurities. The alternative preparation of laurolactam proceeding from cyclododecanone (Beckmann rearrangement) does not give rise to any chlorinated impurities. Nevertheless, yellowing of the polymer formed from this laurolactam is observed.

The colour/yellowing is typically determined by means of a spectrophotometer, with quantification using the b value. In addition, the colour separation ΔE is measured between the polymer before and after ageing. The lower these values, the lower the level of yellowing possessed by the polymer.

### BRIEF SUMMARY OF THE INVENTION

The problem addressed by the present invention was that of providing a process for preparing polymers composed of monomers comprising laurolactam which shows lower yellowing compared to the prior art. The laurolactam was to be obtained via the Beckmann rearrangement, nowadays used customarily on the industrial scale, of the cyclododecanone oxime.

It has now been found that the presence of polycyclic substances having 24 carbon atoms and at least one heteroatom (oxygen, nitrogen) and having a molar mass between 300 and 380 g/mol in laurolactam affects the colour of laurolactam-containing polymers such as nylon-12. Such impurities arise during the preparation of laurolactam and are crucial in causing the yellowing of the polymers after ageing (24 h at 100° C. in an air circulation drying cabinet, air atmosphere).

Accordingly, a process has been found for preparation of polymers from monomers comprising laurolactam, which comprises the following steps:

a. Beckmann rearrangement of cyclododecanone oxime to give laurolactam in the presence of a Beckmann rearrangement catalyst,
b. removal of impurities from the laurolactam to obtain purified laurolactam, and
c. polymerization of monomers comprising purified laurolactam.

### DETAILED DESCRIPTION OF THE INVENTION

The impurities are polycyclic substances comprising 24 carbon atoms and at least one heteroatom selected from nitrogen and oxygen and having a molar mass between 300 and 380 g/mol, wherein the impurities are limited in step b. to 500 ppm, preferably 100 ppm, based in each case on laurolactam. The upper limit is based on the sum total of the above-defined impurities. The impurities preferably have a local absorption maximum in the UV spectrum between 250 nm and 290 nm.

It has been found that, surprisingly, an elevated concentration of these impurities in the laurolactam has a distinct effect on the colour or yellowing of a laurolactam-containing polymer after ageing. Laurolactam-containing polymers that have been prepared by the process according to the invention had a b value of <5 and a ΔE value of <5 after ageing. The values were determined with an X-Rite Color i7 spectrophotometer according to DIN 6174; further details can be found in the examples.

In the context of this invention, polymers composed of monomers comprising laurolactam should be regarded as polymers that are obtained at least from laurolactam as monomer. By definition, monomers other than laurolactam are referred to as comonomers. Thus, the term “polymer composed of monomers comprising laurolactam” encompasses homopolyamides and copolyamides. Polymers composed of monomers comprising laurolactam are especially the homopolyamide nylon-12 or copolyamides such as PEBA or caprolactam-laurolactam copolyamides. Preferred polymers contain at least 20% by weight of laurolactam, more preferably 30% by weight, based in each case on the total weight of the polymer. A particularly preferred polymer is nylon-12.

A suitable Beckmann rearrangement catalyst is selected from an acid having pKa≤0, cyanuric chloride, phosphorus trichloride, thionyl chloride and mixtures thereof, preference being given to sulfuric acid.

The polymerization of the purified laurolactam and any further monomers (comonomers) is preferably effected at a temperature between 250° C. and 350° C. The optional comonomers are required for laurolactam-containing polymers such as copolyamides or polyether-block-amides. The preferred temperature range is between 270° C. and 330° C., more preferably 280° C. to 300° C. The polymerization is preferably undertaken in the presence of water, where the water content is preferably 2% to 51% by weight, based on the total weight of water and monomers. More preferably, 3% to 15% by weight of water is used. In a preferred embodiment, the polymerization is conducted at a temperature between 250° C. and 350° C. in the presence of 2% to 51% by weight of water.

Suitable comonomers are selected from lactams having 6 to 14 carbon atoms, especially caprolactam, amino acids having 6 to 14 carbon atoms, diamines having 4 to 23 carbon atoms, dicarboxylic acids having 4 to 23 carbon atoms and polyethers having two terminal groups. Polyethers may, for example, be polyethylene glycols, polypropylene glycols or polytetramethylene ether glycols. Terminal end groups of the polyethers are, for example, hydroxyl, amino and carboxyl groups. Preferably, the polyethers have two terminal hydroxyl groups, two terminal amino groups, two terminal carboxyl groups or one terminal hydroxyl group and one terminal amino group. A homopolyamide or copolyamide obtained from the process can subsequently be polymerized again with polyethers having two terminal groups.

The polymerization can be conducted by a batchwise method or a continuous method, preference being given to a continuous method. It can be conducted without catalyst or with addition of a catalytic amount of an inorganic acid. Examples of suitable inorganic acids are H₃PO₄ or H₃PO₂.

The impurities that are present after the Beckmann rearrangement can be determined by means of gas chromatography-mass spectrometry (GC-MS). Mass spectrometry enables the determination of the molar mass and the empirical formula. By means of GC, the proportion of impurities in the laurolactam is ascertained. The determinations are conducted with a Triplus Autosampler with Trace GC 1310 and QExactive Orbitrap. The separation was undertaken with a TG 5SilMS column of length 30 m and diameter 0.25 mm. The temperature program was 1 min at 60° C., then heating to 220° C. (8 K/min), then heating to 250° C. (1 K/min) and 10 min at 250° C. MS detection was conducted by means of electron impact ionization within the mass range of 30 to 500 g/mol and a mass resolution R=60 000.

In addition, it was possible by means of UV spectrometry and ¹H NMR and ¹³C NMR to show that at least a large portion of these impurities in the laurolactam have a local absorption maximum between 250 and 290 nm. Moreover, the impurities may include at least one double bond and/or an aromatic ring. UV spectra were measured with a ThermoFisher Scientific Accela 1050 HPLC system. For this purpose, a 200×2 mm Hypersil Gold C18 separation column, particle size 1.9 μm, was utilized and a mixture of water, trifluoroacetic acid, acetonitrile and n-propanol was used as eluent. The photodiode array (FDA) detector of the instrument was used to accumulate UV spectra over the respective peaks and correct the background. NMR spectra were measured with a Bruker 500 MHz spectrometer in CDCl₃.

It is suspected from the proven empirical formulae and the indications from the UV and NMR spectra that the following structures, for example, are impurities in the context of the invention:

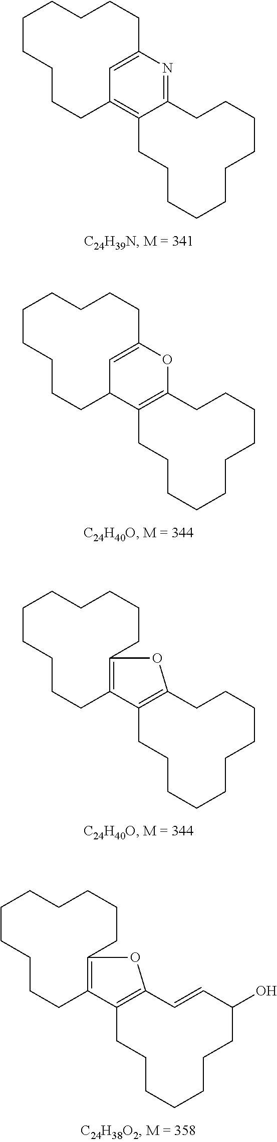 [SMILES: c1c2nc3c(c1CCCCCCCCC2)CCCCCCCCCC3 ; C1=C2CCCCCCCCCC1C1=C(CCCCCCCCCC1)O2 ; C1CCCCCc2c(oc3c2CCCCCCCCCC3)CCCC1 ; OC1/C=C/c2oc3c(c2CCCCCCC1)CCCCCCCCCC3]

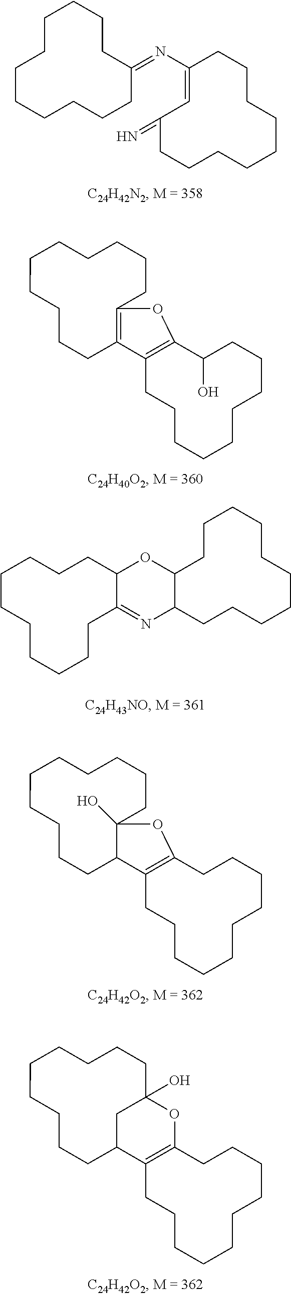 [SMILES: N=C1/C=C(\N=C2CCCCCCCCCCC2)CCCCCCCCC1 ; OC1CCCCCCCCCc2c1oc1c2CCCCCCCCCC1 ; C1CCCCCC2OC3CCCCCCCCCCC3N=C2CCCC1 ; OC12CCCCCCCCCCC1C1=C(CCCCCCCCCC1)O2 ; OC12CCCCCCCCCC(C1)C1=C(CCCCCCCCCC1)O2]

The impurities form as a result of chemical reactions between two compounds containing twelve carbon atoms. These compounds may include, for example, cyclododecanone, cyclododecanone oxime, cyclododecenone (unsaturated ketone) or cyclododecenone oxime (unsaturated oxime). The unwanted side reactions may take place, for example, during the oxime formation, during the Beckmann rearrangement or during the distillation of laurolactam.

The removal of the impurities from the laurolactam (step b.) is preferably undertaken by means of crystallization, distillation or rectification. It is preferable here when distillation is undertaken with at least five theoretical plates.

In a particularly preferred embodiment of the invention, the content of impurities in the laurolactam can additionally be reduced by a workup of cyclododecanone oxime prior to the Beckmann rearrangement. For this purpose, the oxime is subjected to scrubbing with water, scrubbing with alkali such as sodium hydroxide solution, scrubbing with acid such as hydrochloric acid or sulfuric acid, hydrogenation, crystallization or a combination thereof. This treatment of the oxime can reduce the formation of impurities in the laurolactam, such that the purification of the laurolactam subsequently becomes more efficient.

The cyclododecanone oxime is preferably obtained from cyclododecanone. Processes for preparing the oxime are the processes known in the prior art from cyclododecanone and hydroxylamine or hydrogen peroxide with ammonia.

Furthermore, a polymer containing laurolactam, prepared by the process described above, forms a further part of the subject-matter of the invention.

Having generally described this invention, a further understanding can be obtained by reference to certain specific examples which are provided herein for purposes of illustration only, and are not intended to be limiting unless otherwise specified.

### EXAMPLES

The examples which follow describe the preparation of nylon-12 by different processes according to the invention and noninventive reference examples.

The term “impurities” used hereinafter relates to the following definition: polycyclic substances containing 24 carbon atoms and at least one heteroatom selected from oxygen and nitrogen; the molar mass is between 300 and 380 g/mol.

### Examples A: Preparation of Laurolactam

### Example A1: Preparation of Laurolactam with H₂SO₄ and Subsequent Distillation

3.44 kg of a 25% by weight solution of cyclododecanone in hydrocumene were introduced into a 20 l reactor at 90° C. Then 2.83 kg of ammonia solution (25% by weight in water), 171 g of ammonium sulfate, 22 g of Hostapur® (emulsifier), 15.2 g of titanium silicalite TS-1 and 1.84 kg of water were added. The biphasic mixture was stirred vigorously and 550 g of H₂O₂ solution (70% by weight in water) were metered into the reactor over the course of 6 h. At the end, GC analysis showed complete conversion of cyclododecanone to cyclododecanone oxime. The stirring was stopped in order that the phases could separate. The aqueous phase was discharged and the organic phase was washed twice at 95° C. with 5 kg of water.

900 g of concentrated sulfuric acid were then added to the solution of cyclododecanone oxime in hydrocumene from the last step. The biphasic mixture was stirred at 50° C. for 30 min. The phases were then separated. The lower phase comprising cyclododecanone oxime and sulfuric acid was then heated in the reactor at 122° C., attaining a pressure of 1.5 bar. The reaction mixture was stirred for 2 h, in the course of which the cyclododecanone oxime was fully converted to laurolactam. Subsequently, 900 g of cyclododecanone, 1.8 kg of hydrocumene and 2.7 kg of water were added.

After 15 min, the phases were separated. The organic phase was then distilled in a first distillation column having 20 theoretical plates at bottom temperature 200° C. and a pressure of 120 mbar until the bottoms no longer contained any hydrocumene. The bottoms from the first column were subsequently distilled in a second distillation column having 15 theoretical plates at bottom temperature 215° C. and a pressure of 15 mbar until the bottoms no longer contained any cyclododecanone. 850 g of crude laurolactam were obtained in the bottoms from this column. This crude laurolactam had a purity of 99.85% and contained 900 ppm of impurities.

The crude laurolactam was distilled in a third distillation column having 5 theoretical plates at bottom temperature 200° C. and a pressure of 10 mbar. 810 g of laurolactam were obtained in the distillate. The laurolactam had a purity of 99.95% and contained only 30 ppm of impurities.

### Example A2: Preparation of Laurolactam with H₂SO₄ and Subsequent Crystallization

The crude laurolactam from Example A1 was dissolved in 2.85 kg of hydrocumene at 100° C. The resulting 23% by weight solution of laurolactam in hydrocumene was gradually cooled down to 20° C. over 10 h, which led to crystallization of the pure laurolactam. The laurolactam was then filtered off and dried at 100° C. under reduced pressure (1 mbar), which gave 780 g of pure laurolactam. The laurolactam had a purity of 99.99% and contained less than 5 ppm (below the detection limit of the GC instrument) of impurities.

### Example A3: Preparation of Laurolactam with Cyanuric Chloride and Subsequent Distillation

Cyclododecanone oxime was prepared analogously to Example A1.

The solution of cyclododecanone oxime in hydrocumene was dried in the 20 l glass reactor by introduction of N2 at 100° C. over the course of 12 h. Subsequently, the solution had a water content below 100 ppm (Karl Fischer titration). The mixture was heated up to 120° C., and 70 g of a 10% by weight solution of cyanuric chloride in toluene were added. The cyanuric chloride content corresponded to 2 mol % based on cyclododecanone oxime. The mixture was stirred for 2 h, in the course of which the cyclododecanone oxime was fully converted to laurolactam. Subsequently, at 95° C., a 2.5% by weight NaOH solution in water was metered in with vigorous stirring of the biphasic mixture until the pH in the aqueous phase corresponded to pH 11. Then the phases were separated and the aqueous phase was discharged. The organic phase was then washed with a 0.5% by weight H₂SO₄ solution in water until the pH of the aqueous phase was between 6 and 7. The phases were separated and the aqueous phase was discharged.

Subsequently, 900 g of cyclododecanone were added and the mixture was distilled in a first distillation column having 25 theoretical plates at bottom temperature 210° C. and a pressure of 100 mbar until the bottoms no longer contained any hydrocumene or any toluene. The bottoms from the first column were subsequently distilled in a second distillation column having 25 theoretical plates at bottom temperature 190° C. and a pressure of 10 mbar until the bottoms no longer contained any cyclododecanone. 845 g of crude laurolactam were obtained in the bottoms from this column. This crude laurolactam had a purity of 99.46% and contained 1800 ppm of impurities.

The crude laurolactam was distilled in a third distillation column having 15 theoretical plates at bottom temperature 185° C. and a pressure of 5 mbar. 780 g of laurolactam were obtained in the distillate. The laurolactam had a purity of 99.95% and contained only 20 ppm of impurities.

### Examples B: Preparation of Nylon-12

### Example B1 (Inventive): Preparation of Colourless Nylon-12

10 g of pure laurolactam from Example A1 (30 ppm of impurities) were introduced together with 10 g of water, 0.12 g of dodecanedioic acid and 0.6 mg of H₃PO₂ into a 25 ml pressure-rated reactor. The mixture was inertized with nitrogen for 15 min and heated to 130° C. with a metal bath. Then the temperature was heated up to 280° C. over 30 min. This gave rise to a pressure of 10 bar. The temperature was kept at 280° C. for 2.5 h, attaining a pressure of 22 bar. Then the pressure was released gradually down to atmospheric pressure over the course of 30 min. The reaction mixture was blanketed with N₂ at 280° C. for another 2.5 h. The reactor was then cooled down to room temperature, and 10.1 g of nylon-12 were obtained.

After the preparation of the polyamide, on a lathe, the parting tool was used to cut plane-parallel slices (tolerance< 1/10) having a diameter of 14 mm and a thickness of 2 mm from the solidified melt body. Individual slices having cavities were rejected. The colour of the slices was then measured with an X-Rite Color i7 spectrophotometer according to DIN 6174. The measurements were conducted with inclusion of the specular component, with a 10 mm slit and with the D65/10° illuminant. The colour measurement of the polyamide slice gave a b value of 0.3.

The ageing of the samples was conducted in an air circulation drying cabinet under standard atmospheric conditions at a temperature of 100° C. for 24 h. Subsequently, the colour was measured again and the material had a b value of 3.5 and a ΔE value of 4.2.

### Example B2 (Inventive): Preparation of Colourless Nylon-12

10 g of pure laurolactam from Example A2 (less than 5 ppm of impurities) were introduced together with 10 g of water, 0.12 g of dodecanedioic acid and 0.6 mg of H₃PO₂ into a 25 ml pressure-rated reactor. The mixture was inertized with nitrogen for 15 min and heated to 130° C. with a metal bath. Then the temperature was heated up to 280° C. over 30 min. This gave rise to a pressure of 10 bar. The temperature was kept at 280° C. for 2.5 h, attaining a pressure of 22 bar. Then the pressure was released gradually down to atmospheric pressure over the course of 30 min. The reaction mixture was blanketed with N₂ at 280° C. for another 2.5 h. The reactor was then cooled down to room temperature, and 10.1 g of nylon-12 were obtained.

Slices of the polyamide preparation were cut as described above from the solidified melt body. Colour measurement according to DIN 6174 gave a b value of 0.2.

The slice was stored in an air circulation drying cabinet at 100° C. for 24 h. Subsequently, the colour was measured again and the material had a b value of 3.1 and a ΔE value of 4.0.

### Example B3 (Inventive): Preparation of Colourless Nylon-12

10 g of pure laurolactam from Example A3 (20 ppm of impurities) were introduced together with 10 g of water, 0.12 g of dodecanedioic acid and 0.6 mg of H₃PO₂ into a 25 ml pressure-rated reactor. The mixture was inertized with nitrogen for 15 min and heated to 130° C. with a metal bath. Then the temperature was heated up to 280° C. over 30 min. This gave rise to a pressure of 10 bar. The temperature was kept at 280° C. for 2.5 h, attaining a pressure of 22 bar. Then the pressure was released gradually down to atmospheric pressure over the course of 30 min. The reaction mixture was blanketed with N₂ at 280° C. for another 2.5 h. The reactor was then cooled down to room temperature, and 10.1 g of nylon-12 were obtained.

Slices of the polyamide preparation were cut as described above from the solidified melt body. Colour measurement according to DIN 6174 gave a b value of 0.2.

The slice was stored in an air circulation drying cabinet at 100° C. for 24 h. Subsequently, the colour was measured again and the material had a b value of 3.6 and a ΔE value of 4.3.

### Reference Example B4: Preparation of Nylon-12 from Laurolactam with 530 ppm of Impurities

5 mg of mixture of polycyclic substances containing 24 carbon atoms having a molar mass between 300 and 380 g/mol (0.2 mg of C₂₄H₄₀N, 1.5 mg of C₂₄H₄₀O, 1.3 mg of C₂₄H₃₉N, 0.3 mg of C₂₄H₄₃NO, 0.6 mg of C₂₄H₃₈O₂, 0.7 mg of C₂₄H₄₂O₂, 0.4 mg of C₂₄H₄₂O₂) were added to the laurolactam from Example A1. The resulting contaminated laurolactam contained 530 ppm of impurities.

10 g of contaminated laurolactam (530 ppm of impurities) were introduced together with 10 g of water, 0.12 g of dodecanedioic acid and 0.6 mg of H₃PO₂ into a 25 ml pressure-rated reactor. The mixture was inertized with nitrogen for 15 min and heated to 130° C. with a metal bath. Then the temperature was heated up to 280° C. over 30 min. This gave rise to a pressure of 10 bar. The temperature was kept at 280° C. for 2.5 h, attaining a pressure of 22 bar. Then the pressure was released gradually down to atmospheric pressure over the course of 30 min. The reaction mixture was blanketed with N₂ at 280° C. for another 2.5 h. The reactor was then cooled down to room temperature, and 10.1 g of nylon-12 were obtained.

Slices of the polyamide preparation were cut as described above from the solidified melt body. Colour measurement according to DIN 6174 gave a b value of 3.2.

The slice was stored in an air circulation drying cabinet at 100° C. for 24 h. Subsequently, the colour was measured again and the material had a b value of 12.8 and a ΔE value of 9.8. On visual assessment under daylight, the material had a distinctly yellow colour.

### Reference Example B5: Preparation of Nylon-12 from Laurolactam with 520 ppm of Impurities

5 mg of mixture of polycyclic substances containing 24 carbon atoms having a molar mass between 300 and 380 g/mol (0.2 mg of C₂₄H₄₀N, 1.5 mg of C₂₄H₄₀O, 1.3 mg of C₂₄H₃₉N, 0.3 mg of C₂₄H₄₃NO, 0.6 mg of C₂₄H₃₈O₂, 0.7 mg of C₂₄H₄₂O₂, 0.4 mg of C₂₄H₄₂O₂) were added to the laurolactam from Example A3. The resulting contaminated laurolactam then contained 520 ppm of impurities.

10 g of contaminated laurolactam (520 ppm of impurities) were introduced together with 10 g of water, 0.12 g of dodecanedioic acid and 0.6 mg of H₃PO₂ into a 25 ml pressure-rated reactor. The mixture was inertized with nitrogen for 15 min and heated to 130° C. with a metal bath. Then the temperature was heated up to 280° C. over 30 min. This gave rise to a pressure of 10 bar. The temperature was kept at 280° C. for 2.5 h, attaining a pressure of 22 bar. Then the pressure was released gradually down to atmospheric pressure over the course of 30 min. The reaction mixture was blanketed with N₂ at 280° C. for another 2.5 h. The reactor was then cooled down to room temperature, and 10.1 g of nylon-12 were obtained.

Slices of the polyamide preparation were cut as described above from the solidified melt body. Colour measurement according to DIN 6174 gave a b value of 3.0.

The slice was stored in an air circulation drying cabinet at 100° C. for 24 h. Subsequently, the colour was measured again and the material had a b value of 13.0 and a ΔE value of 10.1. On visual assessment under daylight, the material had a distinctly yellow colour.

### Result

Table 1 summarizes the b and ΔE values from the five experiments of the Examples B.

TABLE 1
b and ΔE values from the five experiments

| | | Amount of | | | ΔE |
|---|---|---|---|---|---|
| | Laurolactam | polycyclic | b value | b value | value |
| | from | substances | before | after | after |
| Example | Example . . . | (based on LL) | ageing | ageing | ageing |
| B1 | A1 | 30 ppm | 0.3 | 3.5 | 4.2 |
| (inventive) | | | | | |
| B2 | A2 | 5 ppm | 0.2 | 3.1 | 4.0 |
| (inventive) | | | | | |
| B3 | A3 | 20 ppm | 0.2 | 3.6 | 4.3 |
| (inventive) | | | | | |
| B4 | A1 | 530 ppm | 3.2 | 12.8 | 9.8 |
| (reference) | | | | | |
| B5 | A3 | 520 ppm | 3.0 | 13.0 | 10.1 |
| (reference) | | | | | |

Polyamides which have been prepared from laurolactam having impurities below 500 ppm, after ageing, show low or no yellowish colour by visual assessment. The b and ΔE values thereof are within the range from 3.1 to 4.3 and hence below 5.

By contrast, polyamides that have been synthesized from contaminated laurolactam having impurities above 500 ppm, after ageing, show a visually distinct yellow colour. The b and ΔE values are within a range from 9.8 to 13.0 and hence well above 5.

1. A process for preparing a polymer from at east one monomer comprising laurolactam, the process comprising:
a. performing Beckmann rearrangement of cyclododecanone oxime to give laurolactam in the presence of a Beckmann rearrangement catalyst,
b. removal of at least one impurity from the laurolactam to obtain purified laurolactam, and
c. polymerizing the at least one monomer comprising said purified laurolactam,
wherein the impurity is selected from the group consisting of polycyclic substances containing 24 carbon atoms and at least one heteroatom;
wherein the heteroatom is selected from nitrogen and oxygen;
wherein the polycyclic substances have a molar mass between 300 and 380 g/mol,
wherein the impurities are limited in step b. to 500 ppm, based on laurolactam.

2. The process according to claim 1, wherein the at least one impurity is removed by crystallization or distillation.

3. The process according to claim 2, wherein the distillation is undertaken with at least five theoretical plates.

4. The process according to claim 1, wherein the cyclododecanone oxime is pretreated prior to the Beckmann rearrangement, the pretreatment being selected from the group consisting of scrubbing with water, scrubbing with alkali, scrubbing with acid, hydrogenation, crystallization and combinations thereof.

5. The process according to claim 1, wherein the polymerization is conducted continuously.

6. The process according to claim 1, wherein the Beckmann rearrangement catalyst is selected from an acid with pKA≤0, cyanuric chloride, phosphorus trichloride, thionyl chloride and mixtures thereof.

7. The process according to claim 1, wherein polymerization is conducted at a temperature between 250° C. and 350° C., in the presence of 2% to 51% by weight of water, based on the total weight of monomers and water.

8. The process according to claim 1, wherein the impurities are limited in step b. to 100 ppm, based on laurolactam.

9. The process according to claim 1, wherein the polymer contains at least 20% by weight of laurolactam, based on the total weight of the polymer.

10. A polymer containing laurolactam, prepared according to the process of claim 1.

11. The process according to claim 1, wherein the Beckmann rearrangement catalyst is sulfuric acid.
